(19) United States
(12) Patent Application Publication
(10) Pub. No.: US 2015/0173797 A1
(43) Pub. Date: Jun. 25, 2015

(54) FILLING MATERIAL FOR BONE DEFECT PART

(71) Applicant: HI-LEX CORPORATION, Hyogo (JP)

(72) Inventors: Akihiro Ametani, Hyogo (JP); Kazutaka Yoshino, Hyogo (JP); Takaki Shima, Hyogo (JP); Yasuharu Noishiki, Kanagawa (JP)

(21) Appl. No.: 14/411,724

(22) PCT Filed: Feb. 13, 2013
(86) PCT No.: PCT/JP2013/053364

§ 371 Date: Dec. 29, 2014

(30) Foreign Application Priority Data
Feb. 14, 2012 (JP) 2012-029380

(51) Int. Cl. A61B 17/68; A61L 31/16; A61B 17/84; A61B 17/86; A61B 17/80

(52) U.S. Cl. CPC A61B 17/68; A61B 17/86; A61B 17/8004; A61B 17/842; A61L 31/16; A61B 2017/681

(57) ABSTRACT

To provide a filling material for a bone defect part, which reduces pain and any sense of discomfort and also promotes short-term bone repair. Provided is a filling material for a bone defect part, wherein the filling material is filled in a bone defect part formed between fracture sites in living bone and applies to the fracture site a pressing force in the direction in which the living bone extends from the defect part. Also provided is a filling material for a bone defect part where the filling material is laid in a predetermined length across the bone defect part and continuously applies the pressing force to the fracture site.

### CROSS-REFERENCE TO RELATED APPLICATIONS

This application is the National Stage of International Application No. PCT/JP2013/053364 having International Filing Date, 13 Feb. 2013, which designated the United States of America, and which claims priority from, and the benefit of, Japanese Application No. 2012-029380, filed on 14 Feb. 2012, the disclosures of which are incorporated herein by reference in their entireties.

### TECHNICAL FIELD

The present invention relates to a filling material for a bone defect part to reinforce the bone defect part and promote bone repair.

### BACKGROUND ART

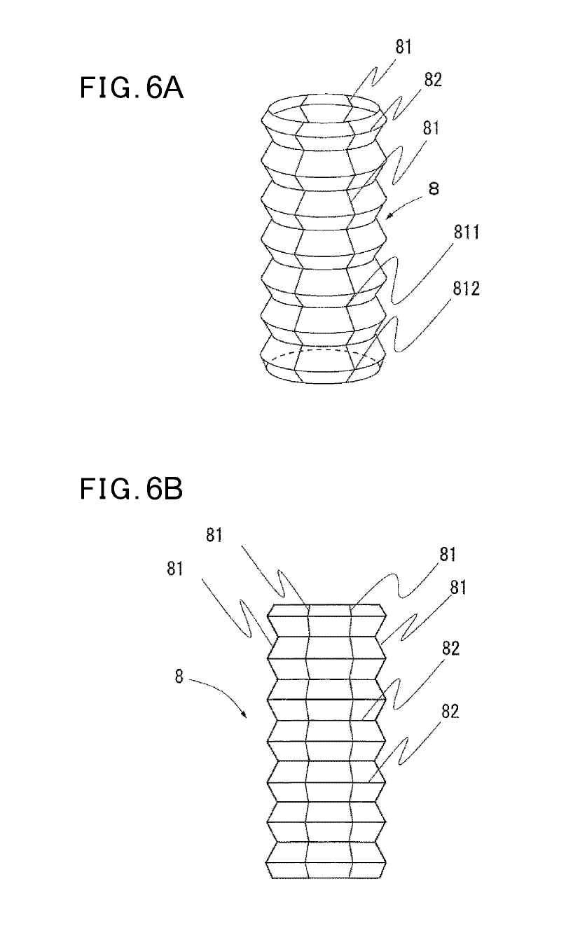

With respect to a filling material for a bone defect part, the following arts are known. Patent Document 1 discloses that a solid support rod is used as a filling material for a bone defect part, introduction layers comprising a coil are provided above and below the support rod, and the introduction layers are connected with both ends of a remaining long bone (Paragraphs [0039] to [0041] and FIG. 6). It discloses that metal such as titanium, ceramic such as alumina, or synthetic resin such as polypropylene, which have a higher rigidity than that of bone, is used as the material of the support rod (Paragraph [0040]). It does not describe that a pressing force is applied to fracture sites of living bone by the materials.

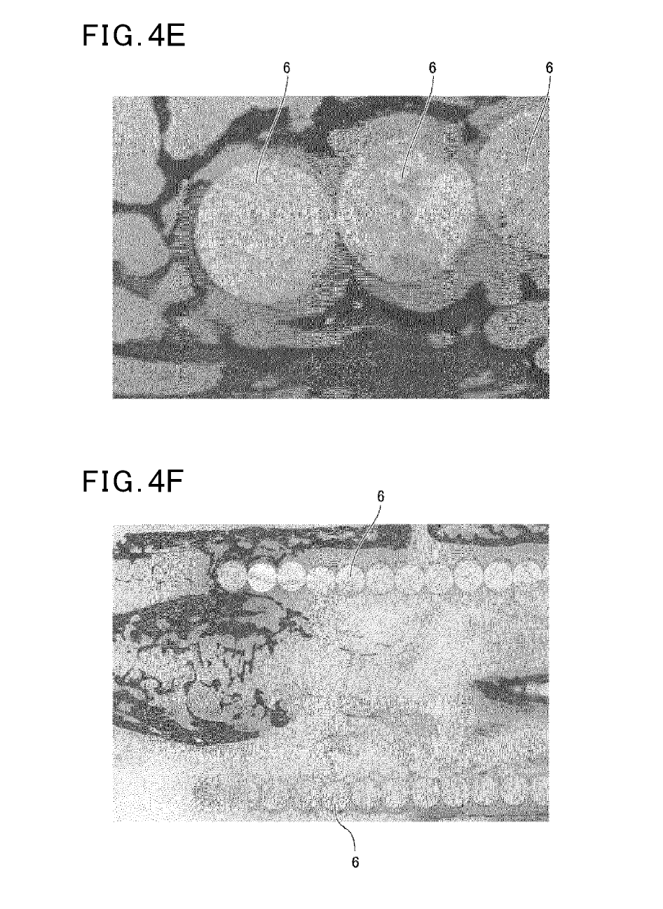

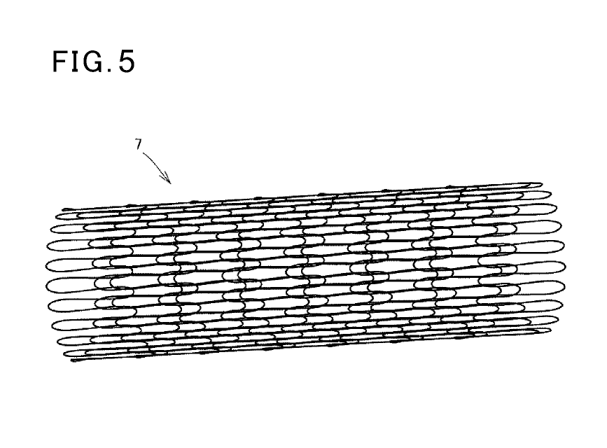

Patent Document 2 discloses that a solid implant material such as an artificial intervertebral disk and bone-filling material for reconstruction of bone defect part is used as a filling material for a bone defect part and the implant material is fixed to a bone defect part, etc. with a coil spring (Paragraph [0004], FIGS. 4 and 5). It discloses that composite porous body of a biodegradable-absorbable polymer, nonporous or porous bioceramic sintered body such as hydroxyapatite and tricalcium phosphate, or metal porous body such as titanium and tantalum, which has a higher rigidity than that of bone, is used as the material of the bone-filling material (Paragraph [0037]). It does not describe that a pressing force is applied to fracture sites of living bone by the materials.

Patent Document 3 and Non-Patent Document 1 disclose a mimics bone (biocompatible implant) formed of a metallic foam material manufactured in combination with steel and aluminum and having elasticity similar to that of bone, and specifically a solid filling material for a bone defect part having a longitudinal elastic modulus similar to that of bone. It does not describe that a pressing force is applied to fracture sites of living bone by the materials.

### PRIOR ART DOCUMENT

### Patent Document 1: JP 2011-015959 A

### Patent Document 2: JP 2010-022387 A

### Patent Document 3: WO 2006/083375

Non-Patent Document 1: Press release of LabSpace (Feb. 16, 2010): “New material mimics bone to create better biomedical implants” http://www.labspaces.net/102021/New_material_mimics_bone_to_create_better_biomedical_implants

### SUMMARY OF THE INVENTION

### Problem to be Solved by the Invention

It is described that the filling materials for the bone defect part of Patent Documents 1 and 2 have a higher rigidity than that of bone, and the filling materials for the bone defect part of Patent Document 3 and Non-Patent Document 1 have a longitudinal elastic modulus similar to that of bone. However, it is not described that a pressing force is applied to fracture sites of living bone by the materials.

Furthermore, the filling materials for the bone defect part of Patent Documents 1 to 3 and Non-Patent Document 1 are solid, and thus cross-sectional structure of bone could not be reproduced inside the filling materials for the bone defect part. Accordingly, prompt tissue repair could not be promoted.

The object of the present invention is to provide a filling material for a bone defect part to solve the above problem.

### Means to Solve the Problem

As a result of keen examination to solve the above problem, the inventors found that pain and any sense of discomfort are reduced significantly and the bone repair is completed in short term by using a filling material for a bone defect part applying a pressing force to fracture sites of living bone, thereby the inventors reach the present invention. Namely, the present invention is a filling material for a bone defect part wherein the filling material for the bone defect part is filled in the bone defect part formed between fracture sites of living bone and applies a pressing force to the fracture site in the direction in which the living bone extends from the defect part. Preferably, the filling material for the bone defect part is laid in a predetermined length across the bone defect part and continuously applies the pressing force to the fracture site.

Examples of the form of the filling material for the bone defect part include bellows, spiral, mesh, and porous forms. If the filling material for the bone defect part of the present invention is hollow, bone marrow and blood vessels enter the hollow portion and grow, and repair of robust tissue can be achieved promptly.

The filling material for the bone defect part of the present invention may contain at least one of factor including bone morphogenetic factor and growth factor, bone affinitive substance, connective tissue affinitive substance, and living cell. They may also be coated to the surface of the filling material for the bone defect part. The coating can be performed by a conventional method. Growth of bone tissue, connective tissue, and periosteum is promoted near the surface of the filling material for the bone defect part by a growth factor, bone affinitive substance, connective tissue affinitive substance, living cell, etc., and repair of tissue can be completed in a shorter period of time. Examples of the applicable growth factor include bone morphogenetic factor (BMP), transforming growth factor-β(TGF-β), insulin-like growth factor (IGF), platelet-derived growth factor (PDGF), fibroblast growth factor (FGF), vascular endothelial cell growth factor (VEGF), epidermal growth factor (EGF), vascular endothelial cell growth factor (VEGF), granulocyte colony-stimulating factor (G-CSF), granulocyte-macrophage colony-stimulating factor (GM-CSF), erythropoietin (EPO), thrombopoietin (TPO), and hepatocyte growth factor (HGF).

Examples of the bone affinitive substance and connective tissue affinitive substance include hydroxyapatite, β-tricalcium phosphate (β-TCP), osteonectin, osteocalcin, bone sialoprotein (BSP), extracellular matrix, collagen, decorin, biglycan, chondroadherin, osteoadherin, chondroitin sulfate, heparan sulfate, dermatan sulfate, and hyaluronic acid. The bone affinitive substance and connective tissue affinitive substance may have the function of bone affinity substance or connective tissue affinity substance, or may have the functions of both bone affinitive substance and connective tissue affinitive substance. An example of the living cell includes osteogenic cell. Namely, osteogenic cell can further be attached to the filling material for the bone defect part of the present invention, for example, by applying a bone marrow liquid to the surface of the filling material for the bone defect part. Alternatively, the filling material for the bone defect part with differentiated osteoblasts can be prepared by culturing mesenchymal stem cell isolated from a bone marrow liquid in vitro and seeding it to the surface of the filling material for the bone defect part and culturing it.

The filling material for the bone defect part may comprise at least one fixation member selected from the group consisting of a rod, a screw, a plate, and a wire to fix the filling material for the bone defect part to the fracture site. For example, if the middle part of a long bone is lost, a rod is inserted into the spongy bone of the long bone at the both sides to fasten the filling material for the bone defect part to both ends of the long bone. As the material of the fixation member, for example, materials used in the filling material for the bone defect part may suitably be used. The filling material and the fixation member may be formed integrally or separately. The filling material for the bone defect part may be configured by a wire-like member.

Examples of the material of the filling material for the bone defect part of the present invention include titanium, stainless, superelastic alloy, shape-memory alloy, biodegradable polymer and the like. With respect to these materials, conventionally known ones or commercially available ones may be used. Examples of the biodegradable-absorbable polymer include poly-L-lactic acid, poly-D,L-lactic acid, copolymer of L-lactic acid and D,L-lactic acid, copolymer of lactic acid and glycolic acid, copolymer of lactic acid and p-dioxanone, copolymer of lactic acid and ethylene glycol, copolymer of lactic acid and caprolactone, and mixtures thereof, which are safe to living bodies. Preferably, the material is one being biocompatible and along the surface of which bone cells can grow and bone tissue, connective tissue, and periosteum can grow. More preferably, the material is titanium.

In the case of bone defect part in long bone, preferably, the filling material for the bone defect part further has flexibility. The “flexibility” refers to one in response to bending stress out of normal stress (compressive stress, tensile stress, and the like), bending stress, torsional stress, and the like acting on the bone and to the property that the material is deflected when it is subjected to bending stress that is below the elastic limit from the side, and it returns to the original shape when the bending stress is removed. The degree of flexibility is measured by the degree of the deflection. In this connection, in conventional filling material for a bone defect part, bending stress has not been considered much. The flexibility can reduce release force to a junction between the filling material for the bone defect part and bone, thereby pain and any sense of discomfort are reduced and bone repair is achieved in short term. It is preferable that the filling material for the bone defect part has more flexibility by 5 to 25% than the bone in an application site of the present invention. The flexibility of the filling material for the bone defect part of the present invention within the target range can be obtained by adjusting the material and the configuration to be used depending on its combination. For example, the configuration of the filling material for the bone defect part of the present invention may be spiral. When the spiral configuration is used, the target flexibility can be obtained, for example, by adjusting spring index (average coil diameter D/wire diameter d) and pitch angle. Although appropriate adjustments are needed depending on the property of a material used, the spring index (average coil diameter D/wire diameter d) can normally be selected from the range of 4 to 30, and preferably, from the range of 8 to 14. The pitch angle is preferably 10° or less. When the spiral filling material for the bone defect part is used, a surgeon can cut the coil of the filling material for the bone defect part at a medical site according to the length of the bone defect part.

The filling material for the bone defect part of the present invention can be applied to bone defect part of human and animal. Specifically, the filling material for the bone defect part having substantially the same outer shape as that of the bone defect part is prepared, applied to the bone defect part, and fixed. Subsequently, examinations such as X-ray are performed periodically to observe the course of bone repair and confirm complete recovery of damaged bone. The filling material for the bone defect part can be applied to not only bone defect part of mammal such as human, dog, cat, monkey, cattle, horse, pig, elephant, and tiger but also that of bird, reptile, amphibian, fish, and the like. The filling material for the bone defect part can be applied to bone defect part of long bone such as femur, humeruse, ulna, clavicle, and rib, as well as the other bones such as scapula, skull, pelvic bone, and breastbone. The filling material for the bone defect part can be applied to any condition, such as a condition where the middle part of a bone is lost completely and the bone is separated into two parts, and a condition where part of a bone is lost although the overall form of the bone is maintained.

### EFFECTS OF THE INVENTION

The present invention can provide a filling material for a bone defect part that reduces pain and any sense of discomfort and also promotes short-term bone repair. Furthermore, the present invention can provide a filling material for a bone defect part inside which cross-sectional structure of bone can be reproduced.

### BRIEF DESCRIPTION OF THE DRAWINGS

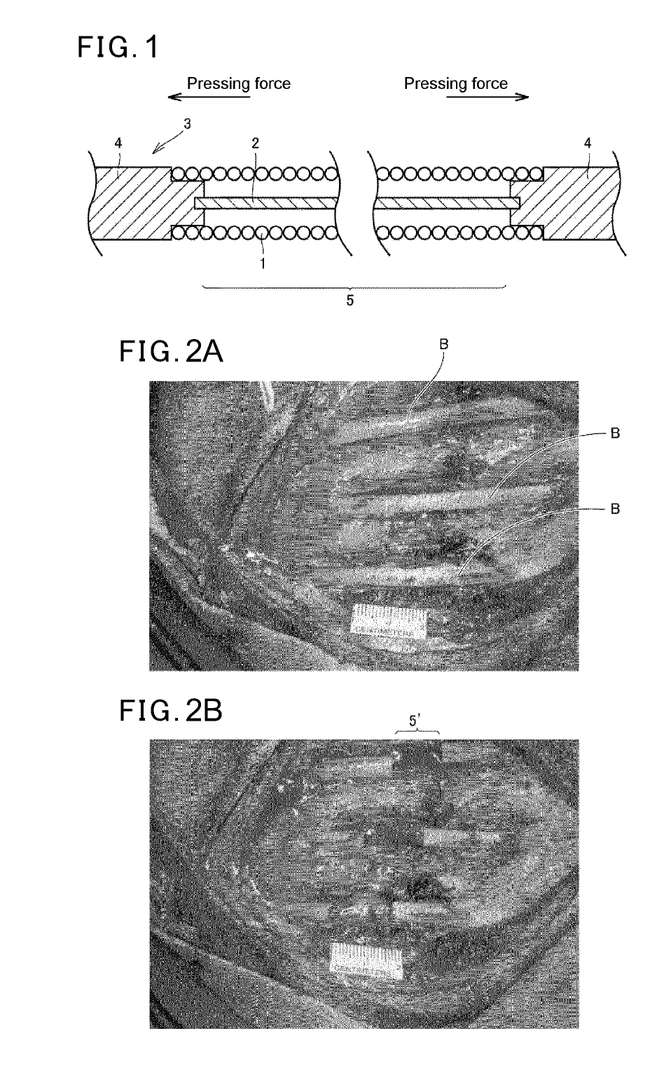

(FIG. 1) A schematic view showing that a spiral filling material for a bone defect part is fastened to bone in an application site using a rod-like fixation member.

(FIG. 2) A photograph showing an experiment using a beagle. Photograph A shows that the chest of the beagle was cut open to expose ribs. Photograph B shows that defect parts of about 30 mm were prepared in the three ribs (in the photograph, the size of the defect parts became about 20 mm with shrinkage of the chest).

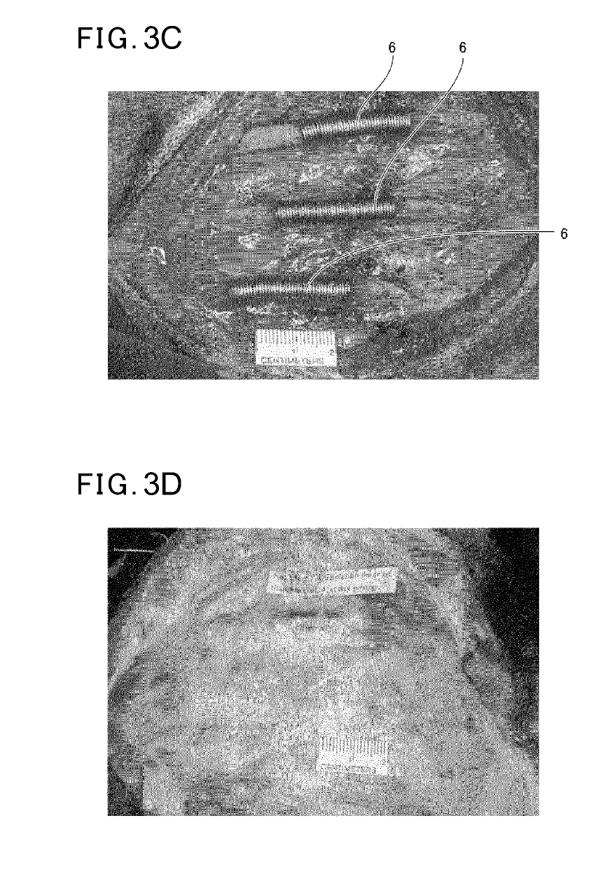

(FIG. 3) A photograph showing an experiment using a beagle. Photograph C shows that spiral filling materials for a bone defect part are fastened to bone defect parts using rod-like fixation members. Photograph D shows the state 41 days after the treatment of Photograph C.

(FIG. 4) A photograph of the bone defect part 115 days after the treatment of Photograph C in FIG. 3. Photograph E is an enlarged photograph in the vicinity of a coil. Photograph F shows the entire bone defect part.

(FIG. 5) A schematic view of a mesh-like filling material for a bone defect part.

(FIG. 6) A is a schematic perspective view of a bellows-like filling material for a bone defect part, and B is a front view of the filling material for the bone defect part of A.

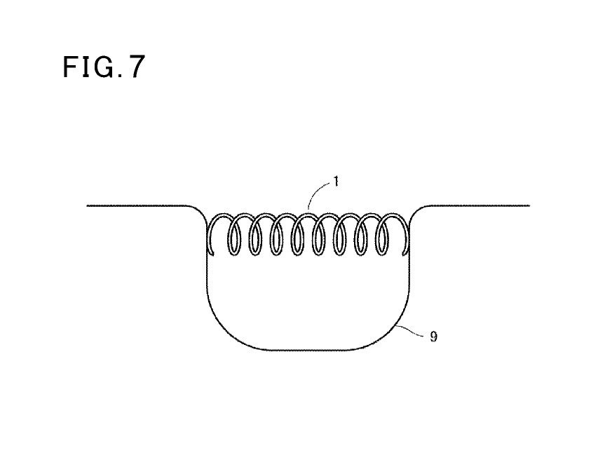

(FIG. 7) A schematic view showing that a filling material for a bone defect part is applied to a recess generated by partial bone defect.

### EMBODIMENT FOR CARRYING OUT THE INVENTION

The present invention will be further described with reference to drawings. FIG. 1 shows an example of repairing a bone defect using a spiral filling material for a bone defect part 1 when the middle part of a long bone 4 is lost completely and the long bone is separated into two parts. In this example, the spiral filling material for the bone defect part 1 is fastened to bone in an application site using a rod-like fixation member 2. As described above, with respect to the spiral filling material for the bone defect part 1 having a substantially the same outer diameter as that of a bone defect part 5, its material, spring index, and pitch angle are set to adjust the flexibility so as to have the flexibility being 5 to 25% higher than that of the lost bone. To fix the spiral filling material for the bone defect part 1, the rod-like fixation member 2 is inserted into the spongy bone of the long bone 4 at the both sides and the spiral filling material for the bone defect part is applied to the bone defect part 5. In this regard, as shown in FIG. 1, it is preferable to cut away outer edges of both ends of the long bone 4 so as to fit the spiral filling material for the bone defect part 1 thereto.

FIGS. 2 to 4 show an outline of an experiment of promoting repair of bone defects of ribs of a beagle using the filling material for the bone defect part of titanium coil of FIG. 1. In Photograph A of FIG. 2, the chest of the beagle was cut open to expose the ribs. In Photograph B, defect parts 5′ of about 30 mm were prepared in the three exposed ribs B, but the size of defect parts 5′ became about 20 mm with shrinkage of the chest. In Photograph C of FIG. 3, the titanium coils 6 are fastened to both ends of the ribs using the filling materials for the bone defect part of titanium coils 6 and rod-like fixation members made of titanium alloy. Photograph D shows the state 41 days after the treatment, and it is understood that inflammation does not occur near the titanium coils 6 and the titanium coils 6 are covered by tissues.

Photographs E and F of FIG. 4 show the bone defect parts 115 days after the treatment. Photograph E is an enlarged photograph in the vicinity of the coil. White portions are connective tissue, and dark portions are bone tissue. The connective tissue covers the periphery of the titanium coil 6 and acts as a cushion, and the bone tissue grows steadily. Photograph F shows the entire titanium coil 6. Bone tissue spreads over the connective tissue outside the titanium coil 6, and periosteum (white portions) spreads outside it. Further, it is understood that so many blood vessels enter inside the titanium coil 6, and tissue formation becomes active.

Furthermore, it has been confirmed by the experiment that the titanium coil 6 bent flexibly to avoid the stress concentration and relax the stress concentration on ends of the bone defect part when excessive force was applied.

Other embodiments of the filling material for the bone defect part of the present invention include those shown in FIGS. 5 to 7.

FIG. 5 is a schematic view of a mesh-like filling material for the bone defect part 1 knitted by stockinet stitch. A knitted body being the filling material for the bone defect part 1 is preferably a knitted body 7 where contacting portions between two lines of the stockinet stitch are fixed. The mesh-like filling material for the bone defect part can apply a pressing force to fracture sites of living bone in the direction in which the living bone extends from the defect part.

FIG. 6 is a schematic view of a bellows-like filling material for the bone defect part. As the filling material for the bone defect part, a bellows-like body 8 shown in FIG. 6A may be used. The bellows-like body 8 is cylindrical as shown in FIG. 6A, and is formed into a mesh shape including longitudinal lines and transversal lines. As the mesh-like and bellows-like body, for example, as shown in FIG. 6B, ring shaped transversal lines 82 are connected to tips of valley parts 811 and mountain parts 812 of zigzag longitudinal lines 81 and the longitudinal lines are fixed to the transversal lines to form the bellows-like and mesh-like body. The manner of connecting the longitudinal lines 81 and the transversal lines 82 is not particularly limited, and the longitudinal lines 81 and the transversal lines 82 are arranged so that the transvresal lines 82 are arranged so as to be positioned outside the cylinder at the valley parts 811, and the transversal lines 82 are arranged so as to be positioned inside the longitudinal lines at the mountain parts 812, and ends of the longitudinal lines are fixed so as to be at a predetermined interval, and thereby the mesh-like body can be formed. The bellows-like body that can be used as the filling material for the bone defect part is not limited to such mesh-like body, and is not particularly limited as long as it is shaped like bellows and can apply a pressing force. If it is such bellows-like body that can generate the pressing force, it can apply the pressing force to fracture sites of living bone in the direction in which the living bone extends from a defect part just like the spiral filling material for the bone defect part.

FIG. 7 shows that the spiral filling material for the bone defect part 1 are mounted to a recess 9 generated by partial bone defect. Examples of the recess 9 include one generated by an accident and the like and one generated when autogenous bone is collected from chin, ilium (hipbone), and the like for autogenous bone graft. For such bone defect part being a recess, the filling material for the bone defect part can apply the pressing force to fracture sites of living bone in the direction in which the living bone extends from the defect part. This promotes a prompt bone repair.

### EXAMPLES

The present invention will be described in more detail by way of example.

### Example 1

### [Filling Material for the Bone Defect Part]

Material: Pure titanium (Product name: TW340, made by Kobe Steel, Ltd.)

Average coil diameter D: 5.8 mm

Wire diameter d: 0.7 mm

Pitch angle: 7.8°

### [Fixation Member]

Material: Titanium alloy (Ti-6Al-4V, made by Kobe Steel, Ltd.) Outer diameter: 1.0 mm

### [Experimental Animal]

Beagles: Six beagles, Female, Age 1.5 to 2.5 years (Weight 10 to 12 kg)

### [Experimental Procedure]

The six beagles are used, their right fifth, sixth, and seventh ribs are exposed by clean operation under general anesthesia, periosteum is separated, and a portion of each rib was removed by 2.5 cm. Then, a titanium rod was inserted into cut ends of each rib by about 5 mm to check whether the titanium rod for fixation of a titanium coil to be an artificial rib can be fitted. After that, the titanium rod was inserted inside the titanium coil, one end of the titanium rod was inserted into one cut end of the rib, the other end of the titanium rod was inserted into the other cut end of the rib, the cut rib was connected through the titanium rod, and the titanium coil was fixed to the cut sites. After that, a bone marrow liquid, β-TCP, bone chips, etc. were infused inside the titanium coil. Finally, an antibiotic was sprinkled, the wound site was covered by a muscular layer, skin suture was performed, and the surgery was completed.

The titanium coil was collected along with the surrounding tissue attached and the rib at postoperative days 41, 115, 148, and 151. After formalin fixation, the collected sample was subjected to soft X-ray. Then, the sample was embedded in resin, 30 μm-thick section was made, bone special staining was performed to observe with an optical microscope.

### [Follow-Up Observation]

In the observation after the surgery, a significant expression of pain was not observed.

### INDUSTRIAL APPLICABILITY

The present invention provides a filling material for a bone defect part that reduces pain and any sense of discomfort and also promotes short-term bone repair. Furthermore, the present invention provides a filling material for the bone defect part inside which bone cross-sectional structure can be reproduced.

### EXPLANATION OF SYMBOLS

1 Filling material for a bone defect part of titanium coil
2 Titanium rod for fixation (fixation member)
3 Bone
4 Long bone
5 Bone defect part
B Rib
5′ Defect part
6 Titanium coil
7 Mesh-like body
8 Bellows-like body
81 Longitudinal line
811 Valley part
812 Mountain part
82 Transversal line
9 Recess

1. A filling material for a bone defect part, wherein the filling material for the bone defect part is filled in the bone defect part formed between fracture sites in living bone and applies a pressing force to the fracture site in the direction in which the living bone extends from the bone defect part.

2. The filling material for the bone defect part according to claim 1, wherein the filling material for the bone defect part is laid in a predetermined length across the bone defect part and continuously applies the pressing force to the fracture site.

3. The filling material for the bone defect part according to claim 1, wherein the filling material for the bone defect part is formed into bellows, spiral, mesh, or porous form.

4. The filling material for the bone defect part according to claim 1, wherein the filling material for the bone defect part is coated by and/or contains one or more kinds selected from the group consisting of factor including bone morphogenetic factor and growth factor, bone affinitive substance, connective tissue affinitive substance, and living cell.

5. The filling material for the bone defect part according to claim 1, wherein the filling material for the bone defect part comprises at least one fixation member selected from the group consisting of a rod, a screw, a plate, and a wire to fix the filling material for the bone defect part to the fracture site.

6. The filling material for the bone defect part of claim 1, wherein the filling material for the bone defect part comprises a wire-like member.
